(19) United States
(12) Patent Application Publication
(10) Pub. No.: US 2024/0156735 A1
(43) Pub. Date: May 16, 2024

(54) Beta-hydroxybutyrate Salt Granule and Methods for Producing the Same

(71) Applicant: NANJING NUTRABUILDING BIO-TECH CO., LTD., Nanjing (CN)

(72) Inventors: Long Jiang, Nanjing (CN); Xuyang Sun, Nanjing (CN); Qiru Fan, Nanjing (CN); Ronghua Yi, Nanjing (CN); Kylin Liao, Nanjing (CN)

(73) Assignee: NANJING NUTRABUILDING BIO-TECH CO., LTD., Nanjing (CN)

(21) Appl. No.: 17/768,409

(22) PCT Filed: Apr. 14, 2021
(86) PCT No.: PCT/CN2021/087134

§ 371 Date: Apr. 12, 2022

(30) Foreign Application Priority Data
Feb. 9, 2021 (WO) PCT/CN2021/076213

(51) Int. Cl. A61K 9/16; A61K 31/19; C07C 59/01

(52) U.S. Cl. CPC A61K 9/1652; A61K 31/19; C07C 59/01

(57) ABSTRACT

Among others, the present invention provides beta-hydroxybutyrate (BHB) salt granules, comprising BHB salts and fillers. The present invention also provides methods for producing BHB salt granules, wherein the BHB salt granules are prepared by granulation process. The production methods may include (a) wet granulation; (b) extrusion; (c) spheronization; and (d) drying.

### FIELD OF THE INVENTION

This invention generally relates to the field of pharmaceutics, and more specifically relates to formulations of beta-hydroxybutyrate (BHB) salt granules and methods for preparing or producing beta-hydroxybutyrate (BHB) salt granules.

### BACKGROUND OF THE INVENTION

Nutritional, or therapeutic, ketosis is the physiological state of elevated blood ketone body levels resulting from ketogenic diets, calorie restriction, therapeutic fasting and/or supplementation with ketogenic precursors. When in ketosis. the body is essentially burning fat for its primary fuel, and begins cleaving fats into fatty acids and glycerol and transforms the fatty acids into acetyl CoA molecules, which are then eventually transformed through ketogenisis into ketone bodies beta-hydroxybutyrate (beta-hydroxybutyrate or “BHB”), acetoacetate (acetylacetonate), and acetone in the liver. BHB and acetoacetate are the ketone bodies used by the body for energy while acetone is removed as a by-product of ketogenesis. Ketone bodies represent alternative energy substrates for both peripheral tissues and the central nervous system.

While there are many health benefits associated with ketosis, it is challenging to pursue or maintain a state of ketosis. Beta-hydroxybutyrate (BHB) salts are pharmaceutics or supplements that contain a ketone (BHB) bound to a mineral. When these BHB salts are consumed, the salt may dissociate into its individual ions and release BHB, which then raises circulating concentrations of BHB in the blood, thereby promoting or sustaining the ketosis state. Notably, conventional BHB salts are as small as dust, and causes many troubles and problems when being processed by downstream manufacturers. In particular, the issues for conventional production methods of BHB salts include, e.g., problem of dust, repeated or prolonged inhalation of dust by workers, high material consumption, and industrial/environmental pollutions.

To overcome these drawbacks, it is therefore desired to have improved BHB salt products, and production methods thereof, with desired form and uniform particle size, to effectively solve the problem of dust, reduce material consumption, reduce industrial pollutions, and reduce the repeated or prolonged inhalation of dust problem of BHB salts.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

This invention generally relates to formulations of BHB salt granules, and methods for producing such BHB salt granules. Particularly, the BHB salt granules according to the present invention have uniform particle size and can effectively solves problem of dust, reduces material consumption, reduces industrial or environmental pollutions, and/or reduces the repeated or prolonged inhalation of dust in association with BHB salts.

One aspect of this invention relates to a beta-hydroxybutyrate (BHB) salt granule, comprising a BHB salt and a filler.

In some embodiments, the BHB salt granule is made of raw materials comprising (a) the BHB salt with a mass percentage ranging from 10% to 99% and (b) the filler with a mass percentage ranging from 1% to 90%.

In some further embodiments, the BHB salt granule is made of raw materials comprising (a) the BHB salt with a mass percentage ranging from 50% to 99%; and (b) the filler with a mass percentage ranging from 1% to 50%.

In some embodiments, the BHB salt comprises a BHB metal salt. For instance, the BHB metal salt may be formed from potassium, calcium, sodium, magnesium, or a mixture thereof (e.g., a mixture of any two or any three thereof, in any ratio).

In some embodiments, the filler comprises microcrystalline cellulose, lactose, mannitol, starch, pregelatinized starch, sodium carboxymethyl cellulose, resistance dextrin, xanthan gum, gum acacia, guar gum, sodium carboxymethyl starch, cross-linked sodium carboxymethyl starch, methyl cellulose, hydroxypropyl methyl cellulose, hydroxyethyl cellulose, β-cyclodextrin, or a mixture thereof (e.g., a mixture of any two or any three thereof, in any ratio).

Another aspect of the present invention provides a composition for promoting and/or sustaining ketosis in a mammal (e.g., human), comprising BHB salt granule(s) as described above.

In a further aspect, the prevent invention relates to a method for producing BHB salt granules, wherein the BHB salt granules are prepared by granulation process.

In some embodiments, the production method comprises (a) wet granulation; (b) extrusion; (c) spheronization; and (d) drying.

In some embodiments, the wet granulation comprises: mixing a BHB salt and a filler; and adding a binder solution for wet granulation to obtain a wet material. For instance, the binder may be water.

In some embodiments, the ratio of the filler and binder ranges from 1:1 to 1:1.5, rotating speed of the wet granulator is 100-500 rounds/minute (“r/min”), and/or the feeding speed is 5-15 r/min.

In some embodiments, the extrusion comprises: extruding the wet material obtained in step (a) by the extruder, to obtain strip material with the same diameter. In some further embodiments, the extruder has sieve plate aperture of 0.8-1.2 mm, and/or the screw speed of the extruder is 100-300 r/min.

In some embodiments, the spheronization comprises: sphering the strip materials obtained in step (b) at a high speed through the spheronization device to get granules with the same size and desired roundness. In some further embodiments, the rotation speed of the spheronization device may be 400-800 r/min, and/or the time of spheronization may be 15-60 s.

In some embodiments, the drying step comprising drying the granules prepared in step (c) to obtain the BHB salt granules. In some embodiments, the moisture content of final material is controlled below 3%. In some further embodiments, the drying temperature may be 40-60° C., and/or the drying time may be 15-30 minutes.

In some embodiments, the BHB salts have a uniform particle size. In some embodiments, the BHB salts have a particle size of 10-100 mesh.

In some embodiments, the BHB salt is formed from potassium, calcium, sodium, magnesium, or a mixture thereof.

In some embodiments, the filler comprises microcrystalline cellulose, lactose, mannitol, starch, pregelatinized starch, sodium carboxymethyl cellulose, resistance dextrin, xanthan gum, gum acacia, guar gum, sodium carboxymethyl starch, cross-linked sodium carboxymethyl starch, methyl cellulose, hydroxypropyl methyl cellulose, hydroxyethyl cellulose, β-cyclodextrin, or a mixture thereof.

Still in some embodiments, the method effectively solves problem of dust, reduces material consumption, reduces industrial or environmental pollutions, and/or reduces the repeated or prolonged inhalation of dust in association with BHB salts.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

### BRIEF DESCRIPTIONS OF THE FIGURES

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1(a) and FIG. 1(b) illustrate the particle size analysis of BHB sodium salts and BHB sodium salt granules according to one embodiment of the present invention.

FIG. 2(a) and FIG. 2(b) illustrate the particle size analysis of BHB calcium salts and BHB calcium salt granules according to one embodiment of the present invention.

FIG. 3(a) and FIG. 3(b) illustrate the particle size analysis of BHB magnesium salts and BHB magnesium salt granules according to one embodiment of the present invention.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

Generally speaking, various embodiments of the present invention provide for formulations of BHB salt granules, which include BHB salts and fillers, as well as methods for producing BHB salt granules, e.g., by granulation. Specifically, the production methods of BHB salt granules according to the present invention may include (a) wet granulation; (b) extrusion; (c) spheronization; and (d) drying.

The present invention may achieve a number of technical advantages. For instance, the particle size of the BHB salt granules prepared according to the present invention can be 10-100 (e.g., 10-40) mesh. Under this particle size, the dust problem of BHB salts can be effectively solved; the materials consumption of BHB salts can be effective reduced; the environmental pollutions of BHB salts can be effectively reduced; and the repeated or prolonged inhalation of dust problem of BHB salts can be effectively reduced. At the same time, the BHB salt granules may maintain the uniform particle size.

The following examples are illustrative of select embodiments of the present invention and are not meant to limit the scope of the invention.

### Example 1

The formulation of BHB salt granules in Example 1 is made of the raw materials with the respective mass percentages, as shown in Table 1 below.

TABLE 1

| Component name | Mass (kg) | Mass percentage (%) |
|---|---|---|
| BHB sodium salt | 4.95 | 99.00 |
| Microcrystalline cellulose | 0.05 | 1.00 |

The preparation method of such BHB salt granules includes the following steps:
(1) Wet granulation: Mix 4.95 kg of BHB sodium salts and 0.05 kg of microcrystalline cellulose, add 0.05 kg of water for wet granulation to obtain wet materials. The rotating speed of the wet granulator is adjusted to 100 r/min and the feeding speed is 5 r/min;
(2) Extrusion: Extrude the wet material obtained in step (1) by the extruder with the sieve plate aperture of 0.8 mm, to obtain strip materials with the same diameter. The screw speed of the extruder is adjusted to 100 r/min;
(3) Spheronization: Sphere the strip materials obtained in step (2) at the high speed through the spheronization device to get granules of the same size and good roundness. The rotation speed of the spheronization device is adjusted to 400 r/min and the time of spheronization is 60 s;
(4) Drying: Dry the granules prepared in step (3) to get BHB salt granules with the moisture content of 2.8% and the particle size of 10-40 mesh. The drying temperature is adjusted to 40° C. and the drying time is 30 minutes.

### Example 2

The formulation of BHB salt granules in Example 2 is made of the raw materials with the respective mass percentages, as shown in Table 2 below.

TABLE 2

| Component name | Mass (kg) | Mass percentage (%) |
|---|---|---|
| BHB calcium salt | 5.00 | 50.00 |
| Lactose | 5.00 | 50.00 |

The preparation method of such BHB salt granules includes the following steps:
(1) Wet granulation: Mix 5 kg of BHB calcium salt and 5 kg of lactose, and add 5 kg of water for wet granulation to obtain wet material. The rotating speed of the wet granulator is adjusted to 300 r/min and the feeding speed is 10 r/min;
(2) Extrusion: Extrude the wet material obtained in step (1) by the extruder with the sieve plate aperture of 1 mm, to obtain strip materials with the same diameter. The screw speed of the extruder is adjusted to 200 r/min;
(3) Spheronization: Sphere the strip materials obtained in step (2) at the high speed through the spheronization device to get granules with the same size and good roundness. The rotation speed of the spheronization device is adjusted to 600 r/min and the time of spheronization is 30 s;
(4) Drying: Dry the granules prepared in step (3) to get BHB salt granules with the moisture content of 2.7% and the particle size of 10-40 mesh. The drying temperature is adjusted to 50° C. and the drying time is 25 minutes.

### Example 3

The formulation of BHB salt granules in Example 3 is made of the raw materials with the respective mass percentages, as shown in Table 3 below.

TABLE 3

| Component name | Mass (kg) | Mass percentage (%) |
|---|---|---|
| BHB potassium salt | 5.00 | 50.00 |
| Starch | 5.00 | 50.00 |

The above-mentioned preparation method of BHB salt granules includes the following steps:
(1) Wet granulation: Mix 5 kg of BHB potassium salt and 5 kg of starch, and add 5 kg of water for wet granulation to obtain wet material. The rotating speed of the wet granulator is adjusted to 500 r/min and the feeding speed is 15 r/min;
(2) Extrusion: Extrude the wet material obtained in step (1) by the extruder with the sieve plate aperture of 1.2 mm, to obtain strip materials with the same diameter. The screw speed of the extruder is adjusted to 300 r/min;
(3) Spheronization: Sphere the strip materials obtained in step (2) at the high speed through the spheronization device to get granules with the same size and good roundness. The rotation speed of the spheronization device is adjusted to 800 r/min and the time of spheronization is 15 s;
(4) Drying: Dry the granules prepared in step (3) to get BHB salt granules with the moisture content of 2.5% and the particle size of 10-40 mesh. The drying temperature is adjusted to 60° C. and the drying time is 15 minutes.

### Example 4

The formulation of BHB salt granules in Example 4 is made of the raw materials with the respective mass percentages, as shown in Table 4 below.

TABLE 4

| Component name | Mass (kg) | Mass percentage (%) |
|---|---|---|
| BHB sodium salt | 7.00 | 70.00 |
| Microcrystalline cellulose | 3.00 | 30.00 |

The above-mentioned preparation method of BHB salt granules includes the following steps:
(1) Wet granulation: Mix 7 kg of BHB sodium salt and 3 kg of microcrystalline cellulose, and add 4 kg of water for wet granulation to obtain wet material. The rotating speed of the wet granulator is adjusted to 300 r/min and the feeding speed is 10 r/min;
(2) Extrusion: Extrude the wet material obtained in step (1) by the extruder with the sieve plate aperture of 1 mm, to obtain strip materials with the same diameter. The rotation speed of the extruder is adjusted to 200 r/min;
(3) Spheronization: Sphere the strip materials obtained in step (2) at the high speed through the spheronization device to get granules with the same size and good roundness. The screw speed of the spheronization device is adjusted to 600 r/min and the time of spheronization is 30 s;
(4) Drying: Dry the granules prepared in step (3) to get BHB salt granules with the moisture content of 2.6% and the particle size of 40-80 mesh. The drying temperature is adjusted to 60° C. and the drying time is 15 minutes.

### Example 5

The formulation of BHB salt granules in Example 5 is made of the raw materials with the respective mass percentages, as shown in Table 5 below.

TABLE 5

| Component name | Mass (kg) | Mass percentage (%) |
|---|---|---|
| BHB sodium salt | 8.00 | 80.00 |
| Microcrystalline cellulose | 2.00 | 20.00 |

The above-mentioned preparation method of BHB salt granules includes the following steps:
(1) Wet granulation: Mix 8 kg of BHB sodium salt and 2 kg of microcrystalline cellulose, and add 3 kg of water for wet granulation to obtain wet material. The rotating speed of the wet granulator is adjusted to 300 r/min and the feeding speed is 10 r/min;
(2) Extrusion: Extrude the wet material obtained in step (1) by the extruder with the sieve plate aperture of 1 mm, to obtain strip materials with the same diameter. The rotation speed of the extruder is adjusted to 200 r/min;
(3) Spheronization: Sphere the strip materials obtained in step (2) at the high speed through the spheronization device to get granules with the same size and good roundness. The screw speed of the spheronization device is adjusted to 600 r/min and the time of spheronization is 30 s;
(4) Drying: Dry the granules prepared in step (3) to get BHB salt granules with the moisture content of 2.5% and the particle size of 60-100 mesh. The drying temperature is adjusted to 60° C. and the drying time is 15 minutes.

### Example 6

The formulation of BHB salt granules in Example 6 is made of the raw materials with the respective mass percentages, as shown in Table 6 below.

TABLE 6

| Component name | Mass (kg) | Mass percentage (%) |
|---|---|---|
| BHB sodium salt | 7.00 | 70.00 |
| Microcrystalline cellulose | 1.50 | 15.00 |
| Lactose | 1.50 | 15.00 |

The above-mentioned preparation method of BHB salt granules includes the following steps:
(1) Wet granulation: Mix 7 kg of BHB sodium salt, 1.5 kg of microcrystalline cellulose and 1.5 kg of lactose, and add 4 kg of water for wet granulation to obtain wet material. The rotating speed of the wet granulator is adjusted to 300 r/min and the feeding speed is 10 r/min;
(2) Extrusion: Extrude the wet material obtained in step (1) by the extruder with the sieve plate aperture of 1 mm, to obtain strip materials with the same diameter. The rotation speed of the extruder is adjusted to 200 r/min;
(3) Spheronization: Sphere the strip materials obtained in step (2) at the high speed through the spheronization device to get granules with the same size and good roundness. The screw speed of the spheronization device is adjusted to 600 r/min and the time of spheronization is 30 s;
(4) Drying: Dry the granules prepared in step (3) to get BHB salt granules with the moisture content of 2.6% and the particle size of 40-80 mesh. The drying temperature is adjusted to 60° C. and the drying time is 15 minutes.

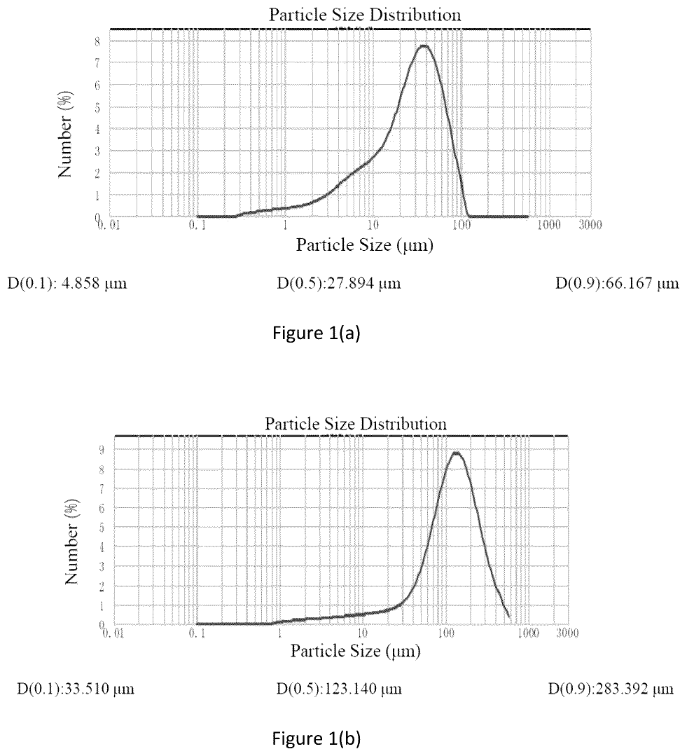

FIG. 1(a) shows the particle size analysis of BHB sodium salts, and FIG. 1(b) shows the particle size analysis of BHB sodium salt granules in Example 6.

### Example 7

The formulation of BHB salt granules in Example 7 is made of the raw materials with the respective mass percentages, as show in Table 7.

TABLE 7

| Component name | Mass (kg) | Mass percentage (%) |
|---|---|---|
| BHB sodium salt | 7.00 | 70.00 |
| Microcrystalline cellulose | 1.50 | 15.00 |
| Starch | 1.50 | 15.00 |

The above-mentioned preparation method of BHB salt granules includes the following steps:
(1) Wet granulation: Mix 7 kg of BHB sodium salt, 1.5 kg of microcrystalline cellulose and 1.5 kg of starch, and add 4 kg of water for wet granulation to obtain wet material. The rotating speed of the wet granulator is adjusted to 300 r/min and the feeding speed is 10 r/min;
(2) Extrusion: Extrude the wet material obtained in step (1) by the extruder with the sieve plate aperture of 1 mm, to obtain strip materials with the same diameter. The rotation speed of the extruder is adjusted to 200 r/min;
(3) Spheronization: Sphere the strip materials obtained in step (2) at the high speed through the spheronization device to get granules with the same size and good roundness. The screw speed of the spheronization device is adjusted to 600 r/min and the time of spheronization is 30 s;
(4) Drying: Dry the granules prepared in step (3) to get BHB salt granules with the moisture content of 2.5% and the particle size of 40-80 mesh. The drying temperature is adjusted to 60° C. and the drying time is 15 minutes.

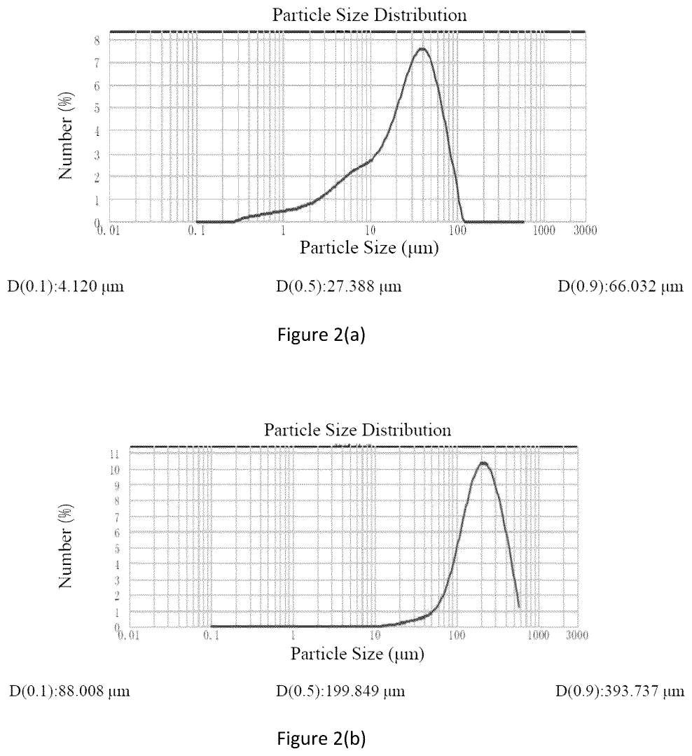

FIG. 2(a) shows the particle size analysis of BHB calcium salts, and FIG. 2(b) shows the particle size analysis of BHB calcium salt granules in Example 7.

### Example 8

The formulation of BHB salt granules in Example 8 is made of the raw materials with the respective mass percentages as shown in Table 8 below.

TABLE 8

| Component name | Mass (kg) | Mass percentage (%) |
|---|---|---|
| BHB sodium salt | 7.00 | 70.00 |
| Lactose | 1.50 | 15.00 |
| Starch | 1.50 | 15.00 |

The above-mentioned preparation method of BHB salt granules includes the following steps:
(1) Wet granulation: Mix 7 kg of BHB sodium salt, 1.5 kg of lactose and 1.5 kg of starch, and add 4 kg of water for wet granulation to obtain wet material. The rotating speed of the wet granulator is adjusted to 300 r/min and the feeding speed is 10 r/min;
(2) Extrusion: Extrude the wet material obtained in step (1) by the extruder with the sieve plate aperture of 1 mm, to obtain strip materials with the same diameter. The rotation speed of the extruder is adjusted to 200 r/min;
(3) Spheronization: Sphere the strip materials obtained in step (2) at the high speed through the spheronization device to get granules with the same size and good roundness. The screw speed of the spheronization device is adjusted to 600 r/min and the time of spheronization is 30 s;
(4) Drying: Dry the granules prepared in step (3) to get BHB salt granules with the moisture content of 2.6% and the particle size of 40-80 mesh. The drying temperature is adjusted to 60° C. and the drying time is 15 minutes.

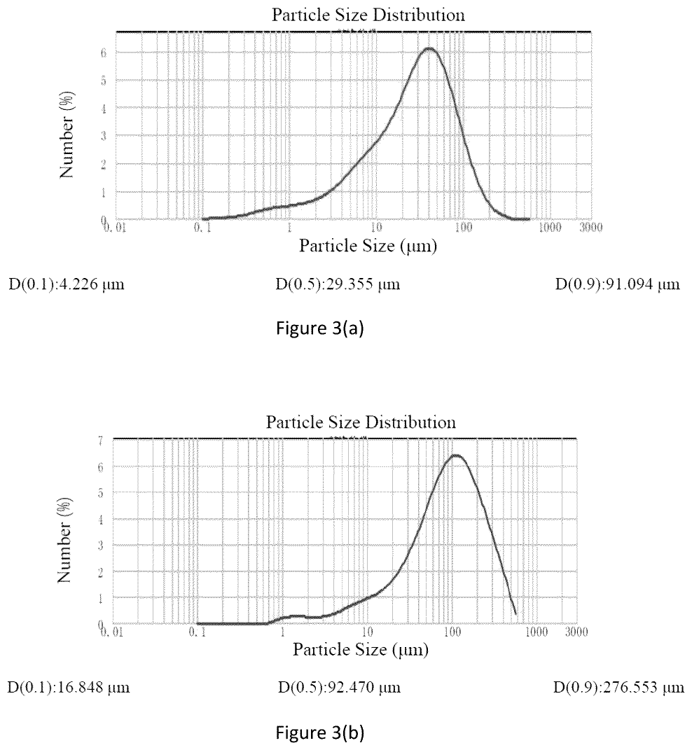

FIG. 3(a) shows the particle size analysis of BHB magnesium salts, and FIG. 3(b) shows the particle size analysis of BHB magnesium salt granules in Example 8.

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

### Other Embodiments

It is to be understood that while the invention has been described in conjunction with the detailed description thereof and accompanying figures, the foregoing description and accompanying figures are only intended to illustrate, and not limit the scope of the invention, which is defined by the scope of the appended claims. Other aspects, advantages, and modifications are within the scope of the following claims. All publications referenced herein are incorporated by reference in their entireties.

1. A beta-hydroxybutyrate (BHB) salt granule, comprising a BHB salt and a filler.

2. The beta-hydroxybutyrate (BHB) salt granule of claim 1, wherein the BHB salt granule is made of raw materials comprising (a) the BHB salt with a mass percentage ranging from 10% to 99%; and (b) the filler with a mass percentage ranging from 1% to 90%.

3. The beta-hydroxybutyrate (BHB) salt granule of claim 2, wherein the BHB salt granule is made of raw materials comprising (a) the BHB salt with a mass percentage ranging from 50% to 99%; and (b) the filler with a mass percentage ranging from 1% to 50%.

4. The beta-hydroxybutyrate (BHB) salt granule of claim 1, wherein the BHB salt comprises a BHB metal salt.

5. The beta-hydroxybutyrate (BHB) salt granule of claim 4, wherein the BHB metal salt is formed from potassium, calcium, sodium, magnesium, or a mixture thereof.

6. The beta-hydroxybutyrate (BHB) salt granule of claim 1, wherein the filler comprises microcrystalline cellulose, lactose, mannitol, starch, pregelatinized starch, sodium carboxymethyl cellulose, resistance dextrin, xanthan gum, gum acacia, guar gum, sodium carboxymethyl starch, cross-linked sodium carboxymethyl starch, methyl cellulose, hydroxypropyl methyl cellulose, hydroxyethyl cellulose, β-cyclodextrin, or a mixture thereof.

7. A composition for promoting and/or sustaining ketosis in a mammal, comprising a BHB salt granule of claim 1.

8. A method for producing BHB salt granules, wherein the BHB salt granules are prepared by a granulation process.

9. The method of claim 8, wherein the granulation process comprises steps of (a) wet granulation; (b) extrusion; (c) spheronization; and (d) drying.

10. The method of claim 9, wherein the wet granulation step comprises
mixing a BHB salt and a filler; and
adding a binder solution for wet granulation to obtain a wet material.

11. The method of claim 10, wherein the binder is water.

12. The method of claim 11, wherein ratio of the filler and binder ranges from 1:1 to 1:1.5 (weight by weight or volume by volume), rotating speed of the wet granulator is 100-500 r/min, and/or feeding speed is 5-15 r/min.

13. The method of claim 8, wherein the extrusion step comprises: extruding the wet material obtained in step (a) by the extruder, to obtain strip material with the same diameter.

14. The method of claim 13, wherein the extruder has an average sieve plate aperture of 0.8-1.2 mm.

15. The method of claim 13 or 11, wherein screw speed of the extruder is 100-300 r/min.

16. The method of claim 8, wherein the spheronization step comprises: sphering the strip materials obtained in step (b) at a high speed through the spheronization device to get granules with the same size and desired roundness.

17. The method of claim 16, wherein rotation speed of the spheronization device is 400-800 r/min, and/or the time of spheronization is 15-60 s.

18. The method of claim 8, wherein the drying step comprising drying the granules prepared in step (c) to obtain the BHB salt granules.

19. The method of claim 18, wherein moisture content of final material is controlled below 3%.

20. The method of claim 18 or 19, wherein drying temperature is 40-60° C., and/or the drying time is 15-30 minutes.

21. The method of claim 8, wherein the BHB salts have a uniform particle size.

22. The method of claim 21, wherein the BHB salts have a particle size of 10-100 mesh.

23. The method of claim 10, wherein the BHB salt is formed from potassium, calcium, sodium, magnesium, or a mixture thereof.

24. The method of claim 10, wherein the filler comprises microcrystalline cellulose, lactose, mannitol, starch, pregelatinized starch, sodium carboxymethyl cellulose, resistance dextrin, xanthan gum, gum acacia, guar gum, sodium carboxymethyl starch, cross-linked sodium carboxymethyl starch, methyl cellulose, hydroxypropyl methyl cellulose, hydroxyethyl cellulose, β-cyclodextrin, or a mixture thereof.

25. The method of claim 8, wherein the method effectively solves problem of dust, reduces material consumption, reduces industrial or environmental pollutions, and/or reduces the repeated or prolonged inhalation of dust in association with BHB salts.
